(19) United States
(12) Patent Application Publication
(10) Pub. No.: US 2025/0223535 A1
(43) Pub. Date: Jul. 10, 2025

(54) PERFUSION CULTURE DEVICE

(71) Applicant: SHANGHAI RUIYU BIOTECH CO., LTD., Shanghai (CN)

(72) Inventors: Ning LI, Shanghai (CN); Xuan WANG, Shanghai (CN); Rui CHEN, Shanghai (CN)

(73) Assignee: SHANGHAI RUIYU BIOTECH CO., LTD., Shanghai (CN)

(21) Appl. No.: 18/406,147

(22) Filed: Jan. 6, 2024

(51) Int. Cl. C12M 1/00; C12M 1/02

(52) U.S. Cl. CPC C12M 29/10; C12M 41/14; C12M 41/20

(57) ABSTRACT

The present disclosure provides a perfusion culture device, wherein the perfusion culture device may comprise an incubator, a heat dissipation assembly and a plurality of power assemblies; the incubator may include a box body and an inner cavity surrounded by the box body, the plurality of power assemblies may be arranged in the inner cavity, the heat dissipation assembly may be fixed on the box body, and the plurality of power assemblies may be arranged on the heat dissipation assembly.

### TECHNICAL FIELD

The present disclosure relates to a field of a medical device, and in particular to a perfusion culture device.

### BACKGROUND

In perfusion culture, after cells and culture fluid are added together to a reactor (e.g., a cell culture mechanism), during a process of cell growth and product formation, some of old culture fluid is continuously removed by a power assembly and new culture fluid is continuously perfused. During a process of cell perfusion culture utilizing a perfusion culture device, a reactor (e.g., a cell culture mechanism) and other components (e.g., a power assembly) are typically placed in an incubator. The heat generated by operations of the power assembly during a delivery process of the culture fluid interferes with the temperature in the incubator, resulting in an unstable culture environment for the cells.

### SUMMARY

One of the embodiments of the present disclosure provides a perfusion culture device. The perfusion culture device may include an incubator, a heat dissipation assembly, and a plurality of power assemblies; wherein the incubator may include a box body and an inner cavity surrounded by the box body, the plurality of power assemblies may be disposed in the inner cavity, the heat dissipation assembly may be fixed on the box body, and the plurality of power assemblies may be arranged on the heat dissipation assembly.

In some embodiments, the plurality of power assemblies may include a drive portion and a working head, the heat dissipation assembly may include an installation bracket and one or more accommodation holes arranged on the installation bracket, the working head may be arranged on the installation bracket, the drive portion may be arranged in the accommodation holes, and at least a portion of inner wall of the accommodation holes may be adhered to the drive portion.

In some embodiments, the heat dissipation assembly may further include one or more fixing members, the one or more fixing members may be connected with the installation bracket respectively. Each of the one or more fixing members may be provided with a corresponding wire slot that may communicate with the one or more accommodation holes.

In some embodiments, the box body may further include a heating assembly. The heating assembly may include a heat-transfer frame and a heating element, wherein the heating element may be arranged on the heat-transfer frame, the installation bracket may be arranged outside the heat-transfer frame, the installation bracket may be arranged adjacent to the heat-transfer frame, and the heat-transfer frame is connected with a bottom wall of the box body to form a culture region.

In some embodiments, the installation bracket may be connected with the bottom wall of the box body; the installation bracket and the heat-transfer frame may form a side wall of the box body; and the installation bracket and the heat-transfer frame may be in an integrated structure.

In some embodiments, the heating assembly may further include one or more dividing members. The one or more dividing members may be connected with the heat-transfer frame and may be arranged within the culture region to divide the culture region into a plurality of sub-culture regions.

In some embodiments, the one or more dividing members may include a dividing portion and two installation portions respectively connected with two ends of the dividing portion. Each of the installation portions may include a connecting plate and a joint plate. The connecting plate may be disposed between the dividing portion and the joint plate and the joint plates of the two installation portions may be lapped onto the heat-transfer frame.

In some embodiments, a plurality of the accommodation holes may be arranged at intervals along a first direction and the plurality of the sub-culture regions may be arranged at intervals along the first direction. A first slot body may be provided on a side of the heat-transfer frame closed to the installation bracket, and a second slot body may be provided on a side of the heat-transfer frame away from the installation bracket. Extension directions of the first slot body and the second slot body may be the same as the first direction. A first heating element may be detachably provided in the first slot body, and a second heating element may be detachably provided in the second slot body.

In some embodiments, the incubator may include an annular cushion layer and an annular heat preservation cover, a top end of the annular heat preservation cover may be connected with a top wall of the box body, and the annular cushion layer may be arranged along a top end of a side wall of the box body. The annular cushion layer may include an annular bottom and an annular retaining portion disposed on the annular bottom, a size of an outer ring of the annular retaining portion may be smaller than a size of an outer ring of the annular bottom, a bottom end of the annular heat preservation cover may be arranged on the annular bottom, and the annular heat preservation cover may be sleeved outside the annular retaining portion.

In some embodiments, the box body may include a first visible heating plate provided on a top wall of the box body and/or a second visible heating plate provided on a bottom wall of the box body.

In some embodiments, the perfusion culture device may further include a ventilation assembly, the ventilation assembly may communicate with the inner cavity, and the ventilation assembly may be configured to exchange gas with the inner cavity.

In some embodiments, the heat dissipation assembly may include an installation bracket and one or more accommodation holes arranged on the installation bracket, and the plurality of power assemblies may be installed in the one or more accommodation holes. The ventilation assembly may include a plurality of ventilation holes, a plurality of ventilation pipes, one or more valves, and a gas pump. Each of the plurality of ventilation holes may communicate with a corresponding ventilation pipe of the plurality of ventilation pipes. Each of the plurality of ventilation holes may form, through the corresponding ventilation pipe, a pathway with the gas pump. The one or more valves may be configured to control a connection or a disconnection of the pathway. The plurality of the ventilation holes may be arranged on the installation bracket. Each of the one or more accommodation holes may be arranged adjacent to at least one of the plurality of ventilation holes, one end of each of the plurality of ventilation holes may be arranged towards the inner cavity, and the other end of each of the plurality of ventilation holes may be arranged towards an outer side of the perfusion culture device.

In some embodiments, the one or more valves may include a switching valve, one end of each of the plurality of ventilation pipes may be connected with a corresponding ventilation hole of the plurality of ventilation holes and the other end of each of the plurality of ventilation pipes may be connected with the switching valve. The switching valve may be connected with the gas pump through a connecting tube, and the switching valve may be configured to control a connection or a disconnection between the connecting tube and the plurality of ventilation pipes.

In some embodiments, the switching valve may include a guide rail and a slider slidingly connected with the guide rail, a plurality of first connecting holes may be arranged side by side along a sliding direction of the slider on the guide rail, the other end of each of the plurality of ventilation pipes may be connected with a corresponding first connecting hole of the plurality of first connecting holes. The slider may be arranged with a second connecting hole, and the second connecting hole may be connected with the connecting tube. The switching valve may be configured to control a sliding position of the slider to change the ventilation pipe connected with the connecting tube or disconnect the connection between the connecting tube and the ventilation pipe.

In some embodiments, the perfusion culture device may further include one or more temperature sensors and a controller, wherein the one or more temperature sensors may be arranged inside the inner cavity, the controller may be connected with the one or more temperature sensors and the ventilation assembly through a signal connection, and the controller may be configured to control the ventilation assembly to exchange the gas with the inner cavity to reduce a temperature of the inner cavity in response to determining that a temperature detected by the one or more temperature sensors exceeds a preset threshold.

In some embodiments, the ventilation assembly may be further configured to control a gas concentration environment within the inner cavity.

In some embodiments, the perfusion culture device may further include a thermal insulating member detachably connected with the box body, wherein the thermal insulating member may divide the inner cavity into a first chamber and a second chamber, the plurality of power assemblies may be arranged in the first chamber, and a culture region of the inner cavity may be arranged in the second chamber.

### BRIEF DESCRIPTION OF THE DRAWINGS

The present disclosure is further illustrated in terms of exemplary embodiments. These exemplary embodiments are described in detail with reference to the drawings. These embodiments are not limited. In these embodiments, the same number represents the same structure, wherein:

FIG. 1 is a schematic diagram illustrating an exemplary structure of a perfusion culture device according to some embodiments of the present disclosure;

FIG. 2 is a schematic diagram illustrating an exemplary exploded view of the perfusion culture device shown in FIG. 1;

FIG. 3 is a schematic diagram illustrating an exemplary installation of a power assembly according to some embodiments of the present disclosure;

FIG. 4 is a schematic diagram illustrating an exemplary structure of a heat dissipation assembly and a heating assembly according to some embodiments of the present disclosure;

FIG. 5 is a schematic diagram illustrating an exemplary structure of a dividing member according to some embodiments of the present disclosure;

FIG. 6 is a schematic diagram illustrating an exemplary internal structure of a heat-transfer frame and a heating assembly according to some embodiments of the present disclosure;

FIG. 7 is a schematic diagram illustrating an exemplary structure of a cushion layer according to some embodiments of the present disclosure;

FIG. 8 is a schematic diagram illustrating an exemplary structure of a ventilation assembly according to some embodiments of the present disclosure; and

FIG. 9 is a schematic diagram illustrating another exemplary structure of a perfusion culture device according to some embodiments of the present disclosure;

Reference signs in accompanying drawings are presented below: 100, perfusion culture device; 110, incubator; 111, inner cavity; 111-1, first chamber; 111-2, second chamber; 112, box body; 113, culture region; 113-1, sub-culture region; 114, heating assembly; 115, heat-transfer frame; 115-1, joint slot; 115-2, first slot body; 115-3, second slot body; 116, heating element; 116-1, first heating element; 116-2, second heating element; 117, dividing member; 1171, dividing portion; 1172, installation portion; 1172-1, connecting plate; 1172-2, joint plate; 118, annular cushion layer; 118-1, annular bottom; 118-2, annular retaining portion; 118-3, projection; 119, annular heat preservation cover; 120, power assembly; 122, drive portion; 124, working head; 130, heat dissipation assembly; 132, installation bracket; 134, accommodation hole; 138, fixing member; 139, wire slot; 141, first heating plate; 142, second heating plate; 160, separation member; 170, ventilation mechanism; 171, ventilation hole; 172, ventilation pipe; 173, valve; 173-1 guide rail; 173-2, slider; 174, gas pump; 175, connecting tube.

### DETAILED DESCRIPTION

The technical schemes of embodiments of the present disclosure will be more clearly described below, and the accompanying drawings need to be configured in the description of the embodiments will be briefly described below. Obviously, the drawings in the following description are merely some examples or embodiments of the present disclosure, and will be applied to other similar scenarios according to these accompanying drawings without paying creative labor. Unless obviously obtained from the context or the context illustrates otherwise, the same numeral in the drawings refers to the same structure or operation. Unless obviously obtained from the context or the context illustrates otherwise, the same numeral in the drawings refers to the same structure or operation.

It should be understood that the “system,” “device,” “unit” and/or “module” used herein is a method for distinguishing different components, elements, components, parts or assemblies of different levels. However, if other words may achieve the same purpose, the words may be replaced by other expressions.

As shown in the present disclosure and claims, unless the context clearly prompts the exception, “a,” “one,” and/or “the” is not specifically singular, and the plural may be included. Generally, the terms “including” and “comprising” suggest only the inclusion of clearly identified steps and elements, which do not constitute an exclusive list, and other steps or elements may be included in the method or apparatus.

During a perfusion culture process, some of the old culture fluid may be continuously removed from a reactor (e.g., a cell culture mechanism) and new culture fluid may be continuously perfused by a power assembly. However, the power assembly may generate heat during the operation and gather near the power assembly, which causes local overheating within the perfusion culture device, resulting in uneven temperature distribution and an unstable temperature environment within the perfusion culture device, thus affecting the culture effect of the cells.

The present disclosure describes a perfusion culture device, mainly including an incubator, a heat dissipation assembly, and a plurality of power assemblies. The incubator may include a box body and an inner cavity surrounded by the box body, the plurality of power assemblies may be arranged in the inner cavity, and the heat dissipation assembly may be arranged on the box body. The plurality of power assemblies may be arranged on the heat dissipation assembly, and the heat dissipation assembly may be configured to rapidly dissipate the heat generated by the operations of the power assemblies to prevent the heat from gathering in a vicinity of the power assemblies, which can avoid local overheating in the inner cavity and provide a more uniform and stable temperature environment for the perfusion culture. Moreover, an arrangement of the heat dissipation assembly also avoids a situation in which the working performance of the power assemblies is adversely affected or even the power assemblies are damaged due to the accumulation of heat.

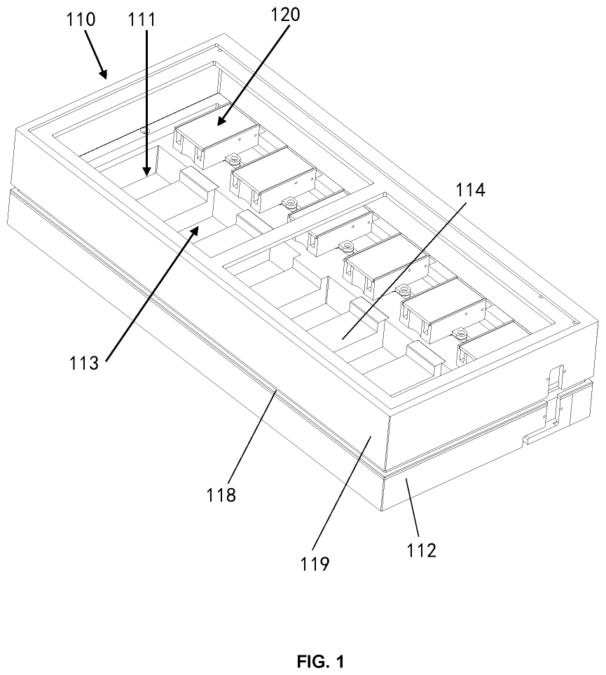

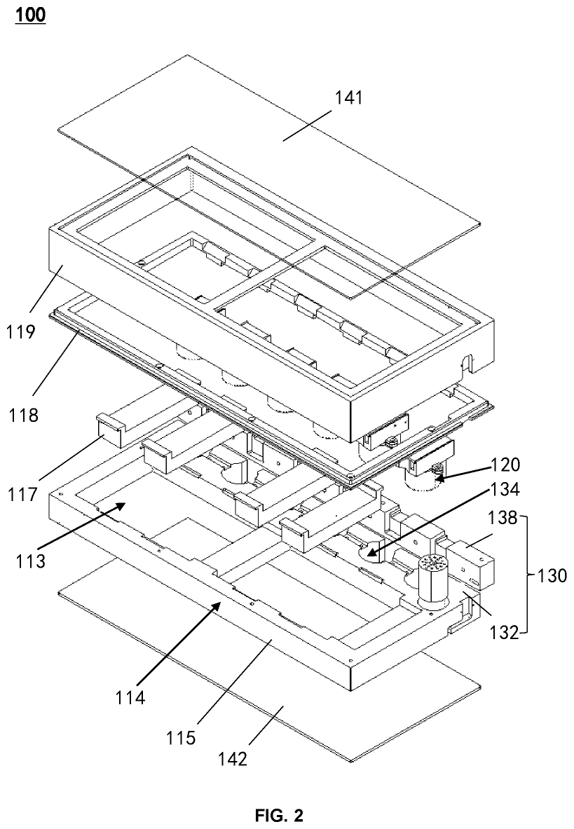

FIG. 1 is a schematic diagram illustrating an exemplary structure of a perfusion culture device according to some embodiments of the present disclosure. FIG. 2 is a schematic diagram illustrating an exemplary exploded view of the perfusion culture device shown in FIG. 1. As shown in FIG. 1 and FIG. 2, the perfusion culture device 100 primarily may include an incubator 110, a plurality of power assemblies 120, and a heat dissipation assembly 130. The incubator 110 may include a box body 112 and an inner cavity 111 surrounded by the box body 112, and the inner cavity 111 may be configured to provide a space and a desirable culture environment (e.g., a temperature environment, a humidity environment, and a gas concentration environment, etc.) for perfusion culture. The box body 112 may separate the inner cavity 111 from an external environment to reduce an impact of external factors on the environment within the inner cavity 111, so that the inner cavity 111 maintains a more stable environment. The plurality of power assemblies 120 may be arranged in the inner cavity 111, and the power assemblies 120 are configured to provide power for transferring the culture fluid during the perfusion culture process. The heat dissipation assembly 130 may be arranged on the box body 112, and the plurality of power assemblies 120 may be arranged on the heat dissipation assembly 130. The heat dissipation assembly 130 may rapidly dissipate the heat generated by the operation of the power assemblies 120 to prevent local overheating within the inner cavity 111, which not only enables the temperature environment within the inner cavity 111 to be uniformly distributed and stabilized, but also avoids a situation in which the working performance of the power assemblies 120 is adversely affected or even the power assemblies 120 are damaged due to the accumulation of heat.

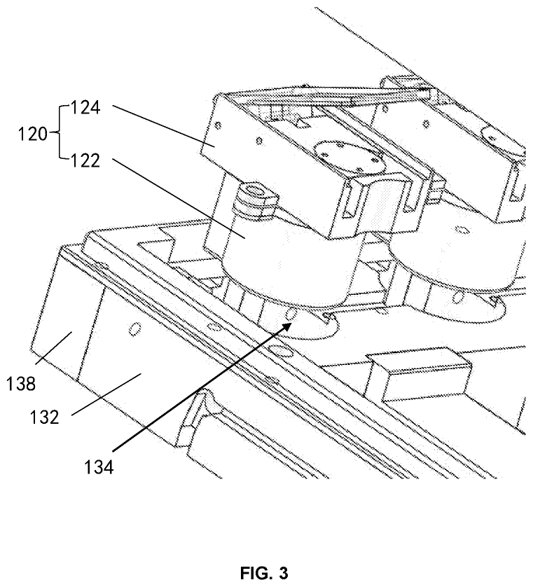

FIG. 3 is a schematic diagram illustrating an exemplary installation of a power assembly according to some embodiments of the present disclosure. As shown in FIG. 3, each of the plurality of power assemblies 120 may include a drive portion 122 and a working head 124, and the drive portion 122 may be configured to drive the working head 124 to work, which enables the working head 124 to output power. In some embodiments, the perfusion culture device may further include a delivery line (not shown in the figures) in which the culture fluid may flow, and the working head 124 may match the delivery line to drive a transfer of the culture fluid within the delivery line.

The drive portion 122 is a power portion of the power assemblies 120 for converting electrical energy, chemical energy, or other mechanical energy capable of being stored and released into mechanical energy that drives the working head 124 to work. In some embodiments, the drive portion 122 may include a device that provides a power output, such as a motor, a fuel engine, or a pneumatic engine.

In some embodiments, the drive portion 122 may include a motor. In some embodiments, an operating voltage of the drive portion 122 may be 3-24 V, such as 5 V. In some embodiments, a power of the drive portion 122 may be 1-30 W, such as 1.1 W.

The working head 124 is a working portion of the power assemblies 120 for powering the transfer of the culture fluid in the delivery line. In some embodiments, the working head 124 may include a peristaltic pump head, a piston pump head, a centrifugal pump head, a jet pump head, or the like. It should be noted that the working head 124 is generally a miniature pump head (e.g., a peristaltic pump head, a syringe pump head, etc.) due to a high flow rate accuracy required for the culture fluid in perfusion culture. For example, when the working head 124 is a peristaltic pump head, the delivery line may be a hose, the culture fluid may flow in the hose, and the hose may match the peristaltic pump head, and the drive portion 122 may drive a roller of the peristaltic pump head to roll and extrude the hose, so that the culture fluid in the hose may be moved along a direction of the rolling of the roller to provide the power for the transfer of the culture fluid. As another example, when the working head 124 is a syringe pump head, the delivery line may be connected with the syringe pump head, and the syringe pump head may include members such as a screw, a piston, an injection head, and the drive portion 122 may drive the screw to move to change a rotary motion into a linear motion, and the screw may push the piston to move, so as to make the culture fluid output from the injection head and carry out the delivery of the culture fluid.

In some embodiments, each of the power assemblies 120 may include one or more drive portions 122 and one or more working heads 124. For each of the power assemblies 120, a count of the drive portions 122 has a predetermined correspondence with a count of the working heads 124. For example, the count of the drive portions 122 may be the same as the count of the working heads 124, and each of the drive portions 122 may provide a work power for a working head 124, respectively. As another example, a drive portion 122 may correspond to a plurality of working heads 124, and a drive portion 122 may provide the work power to the plurality of working heads 124 at the same time. As a further example, a plurality of drive portions 122 may correspond to a working head 124, and the plurality of drive portions 122 may collectively provide the working power to one working head 124.

In some embodiments, a count of power assemblies 120 may be one or multiple, and a user may determine the count of power assemblies 120 based on actual needs, and determine the count of drive portions 122 and working heads 124 included in each power assembly 120. In some embodiments, when the inner cavity 111 serves as an integral culture region and when a requirement of a transfer rate of the culture fluid is not to be high, only one power assembly 120 may be used to provide the power to the transfer of the culture fluid, and the power assembly 120 may include only one drive portion 122 and one working head 124. In some embodiments, when a portion of the inner cavity 111 is used as a culture region, a plurality of power assemblies 120 may be configured at a corresponding position according to a region to be used in the portion to save costs. At this point, each power assembly 120 may include two drive portions 122 and two corresponding working heads 124. In some embodiments, when the inner cavity 111 is divided into a plurality of different culture regions, a corresponding count of power assemblies 120 may be separately provided according to each of the different culture regions individually to ensure an independence of delivery of the culture liquid for each culture region. The count of drive portions 122 and working heads 124 included in the power assembly 120 corresponding to each culture region may be different. For example, when the inner cavity 111 is divided into two regions A and B, three power assemblies 120 may be provided in the region A, and each power assembly 120 in the region A may include one drive portion 122 and two working heads 124, respectively; and four power assemblies 120 may be provided in the region B, and each power assembly 120 in the region B may include two drive portions 122 and one working head 124, respectively.

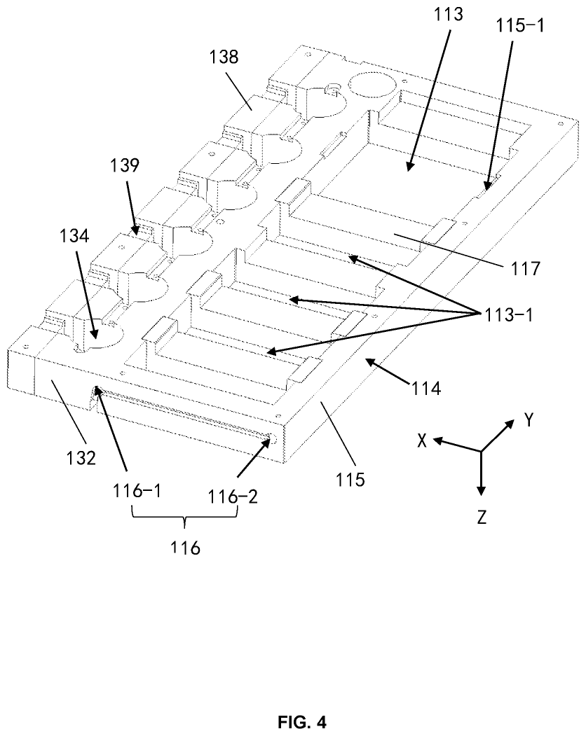

FIG. 4 is a schematic diagram illustrating an exemplary structure of a heat dissipation assembly and a heating assembly according to some embodiments of the present disclosure. As shown in FIG. 3 and FIG. 4, in some embodiments, the heat dissipation assembly 130 may be arranged in the inner cavity 111 for rapidly dissipating the heat generated by the power assembly 120 for the purpose of dissipating heat and cooling down the power assembly 120 to avoid accumulation of heat at the power assembly 120.

The heat dissipation assembly 130 may include an installation bracket 132, and the installation bracket may be configured to install the power assembly 120. In some embodiments, the installation bracket 132 may be provided with one or more accommodation holes 134 to accommodate the drive portion 122. A shape of the accommodation hole 134 may match the drive portion 122 to increase a contact area between an inner wall of the accommodation hole 134 and the drive portion 122 to enhance a heat transfer rate between the drive portion 122 and the installation bracket 132.

In some embodiments, the installation bracket 132 may enclose with the box body 112 to form an inner cavity 111, i.e., the heat dissipation assembly 130 may be arranged outside the inner cavity 111. At this time, the one or more accommodation holes 134 may be regarded as being arranged outside the inner cavity 111. The drive portion 122 of the power assembly 120 arranged in the accommodation hole 134 may be located outside the inner cavity 111, and the working head 124 arranged on the installation bracket 132 may be located inside the inner cavity 111, which can prevent the heating of the drive portion 122 from affecting a temperature environment of the inner cavity 111 without laying the delivery line outside the box body 112, resulting in simpler arrangement of the delivery line, avoiding the external gas from dissolving into the culture fluid through a wall of the delivery line, thus making the concentration control of the gas in the inner chamber 111 more accurate.

In order to avoid an accumulation of heat generated by the operation of the drive portion 122 and enable the heat generated by the operation of the drive portion 122 to be quickly dissipated, in some embodiments, a material of preparing the installation bracket 132 may include a thermally conductive material, such as silver, aluminium alloy, or copper (e.g., pure copper), or the like, which can enhance the heat transfer rate of the installation bracket 132.

In some embodiments, the working head 124 may be installed on the installation bracket 132 and the drive portion 122 may be arranged within the accommodation hole 134. At least a portion of the inner wall of the accommodation hole 134 may be adhered to the drive portion 122 such that heat generated by the operation of the drive portion 122 may be quickly transferred to the installation bracket 132 through the inner wall of the accommodation hole 134 and emitted through the installation bracket 132.

In some embodiments, the accommodation hole 134 may be a blind hole opened downward from a top surface of the installation bracket 132 (e.g., a Z-direction in FIG. 4, wherein the Z-direction may be a height direction of the heat-transfer frame 115), a bottom surface of the accommodation hole 134 may be at least partially adhered with a bottom surface of the drive portion 122, and an inner sidewall of the accommodation hole 134 may be at least partially adhered with an outer sidewall of the drive portion 122, in order to increase a contact area between the drive portion 122 and the accommodation hole 134, thereby increasing the heat transfer rate between the drive portion 122 and the installation bracket 132. For example, the contact area between the drive portion 122 and the accommodation hole 134 is maximum when the bottom surface of the accommodation hole 134 is fully adhered with the bottom surface of the drive portion 122 and the inner sidewall of the accommodation hole 134 is fully adhered with the outer sidewall of the drive portion 122. In some embodiments, the accommodation hole 134 may also be a through-hole opened downward (e.g., the Z direction in FIG. 4) from the top surface of the installation bracket 132, and the inner sidewall of the accommodation hole 134 may be at least partially adhered with the outer sidewall of the drive portion 122. In some embodiments, when the inner wall of the accommodation hole 134 is a discontinuous surface (e.g., a discontinuous curved surface or a discontinuous flat surface), at least a portion of the inner sidewall of the accommodation hole 134 may be adhered the outer sidewall of the drive portion 122.

In some embodiments, a count of accommodation holes 134 is greater than or equal to the count of power assemblies 120 to ensure that each power assembly 120 is capable of being installed on the installation bracket 132. For example, the count of accommodation holes 134 may be the same as the count of power assemblies 120, and each of the accommodation holes 134 may correspond to a drive portion 122 and a working head 124, respectively. As another example, the count of accommodation holes 134 may also be greater than the count of power assemblies 120, and the plurality of power assemblies 120 may be arranged in different accommodation holes 134 continuously or at intervals. The count of accommodation holes 134 may be determined according to actual needs, which may not be limited herein.

In some embodiments, the heat dissipation assembly 130 may also include one or more fixing members 138 connected with the installation bracket 132, the fixing member 138 may increases a contact area between the working head 124 and the heat dissipation assembly 130, and the fixing member 138 may assist the installation bracket 132 in dissipating heat. The fixing member 138 may match the installation bracket 132 to install and fix the drive portion 122 (e.g., the working head 124 of the drive portion 122) of the power assembly 120, and at the same time, the fixing member 138 may match the installation bracket 132 to provide a support for the working head 124 of the power assembly 120. In some embodiments, the fixing member 138 may be a strip (as shown in FIG. 4), and the fixing member 138 may match the installation bracket 132 and fix the plurality of power assemblies 120. In some embodiments, the fixing member 138 may also be a block, and the count of the fixing members 138 may be multiple, and each fixing member 138 may correspond to a power assembly 120.

In some embodiments, the fixing member 138 may be fixedly connected with the installation bracket 132, e.g., by bonding, welding, or the like. In some embodiments, the fixing member 138 may be detachably connected with the installation bracket 132, e.g., snap-fitting, threaded connection, or the like. In some embodiments, the fixing member 138 and the installation bracket 132 may also be in an integrated structure.

In some embodiments, the material used to prepare the fixing member 138 may include a thermally conductive material, such as silver, aluminum alloy, or copper (e.g., pure copper), so that the heat generated by the operation of the power assembly 120 may be quickly transferred to the fixing member 138 through the installation bracket 132 and emitted to the outside world through the fixing member 138. In some embodiments, the fixing member 138 and the installation bracket 132 may be made of the same or different materials.

In some embodiments, each of the one or more fixing members 138 may be provided with a wire slot 139 that may communicate with the one or more accommodation holes 134. The wire slot 139 may provide access to a wiring of the power assembly 120.

Referring to FIG. 1 and FIG. 2, in some embodiments, a shape of the box body 112 may be various, such as a cube, a cylinder, or other arbitrary shapes. In some embodiments, the box body 112 may generate heat to maintain the environment within the inner cavity 111 in a certain temperature range to provide suitable conditions for perfusion culture.

In some embodiments, the box body 112 may include a heating assembly 114, and the heating assembly 114 may generate heat to adjust and keep a temperature within the inner cavity 111, so that the temperature within the inner cavity 111 is maintained within a certain range, thereby providing a suitable temperature environment for the perfusion culture.

The heating assembly 114 may include a heat-transfer frame 115 and a heating element 116. The heat-transfer frame 115 and the bottom wall of the box body 112 may be connected to form the culture region 113, and the heating element 116 may be arranged on the heat-transfer frame 115. The heating element 116 may be a heat generator for generating the heat, and the heat-transfer frame 115 may disperse the heat generated by the heating element 116 to different positions of the culture region 113, so that the temperature of the culture region 113 is more uniformly distributed while being able to be stably maintained within a certain range. In some embodiments, the heating element 116 may include but is not limited to, one or more of a resistive heater, an induction heater, or an infrared heater capable of converting other energy sources into heat.

As shown in FIG. 1, FIG. 2, and FIG. 4, in some embodiments, the installation bracket 132 may be arranged on a side of the heat-transfer frame 115, the installation bracket 132 may be connected with the bottom wall of the box body 112, and the installation bracket 132 and the heat-transfer frame 115 may form a sidewall of the box body 112. In some embodiments, the installation bracket 132 and the heat-transfer frame 115 may be in an integrated structure to reduce a medium between the installation bracket 132 and the heat-transfer frame 115, and enhance a heat transfer rate between the installation bracket 132 and the heat-transfer frame 115. In some embodiments, the installation bracket 132 and the heat-transfer frame 115 may also be a split structure that is connected with each other.

In some embodiments, a material of the heat-transfer frame 115 may include a thermally conductive material, such as silver, aluminium alloy, or copper (i.e., pure copper). In some embodiments, the material of the heat-transfer frame 115 may be the same or different from the material of the installation bracket 132.

In some embodiments, the heat generated by the drive portion 122 of the power assembly 120 may be transferred to the installation bracket 132, and then dispersed to the culture region 113 through the heat-transfer frame 115. The heat generated by the operation of the drive portion 122 may be recovered and reused through the heating assembly 114 which on the one hand avoids the accumulation of heat at the drive portion 122 leading to a local overheating of the inner cavity 111, on the other hand recycles the heat to maintain the temperature of the culture region 113, so as to achieve a more uniform distribution of the temperature in the culture region 113, and to save energy.

In some embodiments, a thermal insulation layer (not shown in the figure) may be arranged between the installation bracket 132 and the heat-transfer frame 115. The thermal insulation layer can effectively prevent heat transfer from the heat-transfer frame 115 to the installation bracket 132, thereby reducing heat dissipation from the heat-transfer frame 115. At the same time, the thermal insulation layer can also prevent excessive heat accumulation within the installation bracket 132 during the operation of the plurality of power assemblies 120, avoiding local overheating and heat transfer to the heat-transfer frame 115, which affects temperature stability of the inner cavity 111.

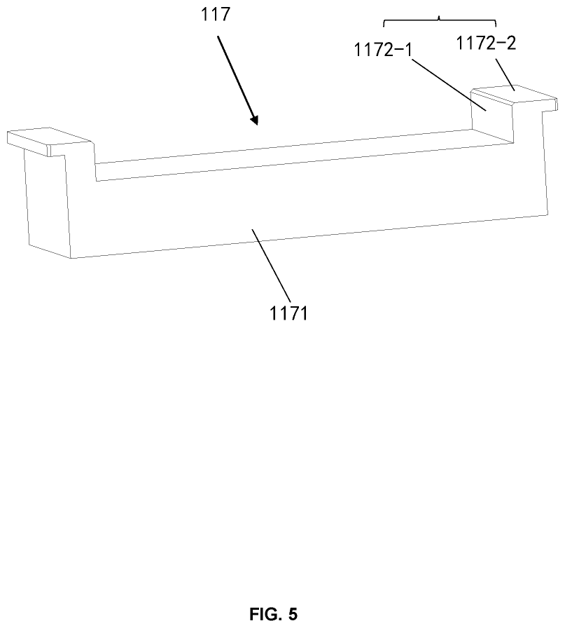

FIG. 5 is a schematic diagram illustrating an exemplary structure of a dividing member according to some embodiments of the present disclosure. As shown in FIG. 4 and FIG. 5, the heating assembly 114 may also include one or more dividing members 117. The one or more dividing members 117 may be connected with the heat-transfer frame 115, and the one or more dividing members 117 may be arranged within the culture region 113 to divide the culture region 113 into a plurality of sub-culture regions 113-1, and the plurality of sub-culture regions 113-1 may be separately used for conducting perfusion culture respectively to enhance the scale of culture. In some embodiments, the dividing member 117 may also be made of a heat-transfer material (e.g., copper, etc.), thereby enhancing the stability and uniformity of the temperature of the plurality of sub-culture regions 113-1.

In some embodiments, corresponding culture containers may be placed in the culture region 113 and the sub-culture region 113-1 according to space sizes of the culture region 113 and the sub-culture region 113-1. For example, a culture plate (e.g., a 96-well culture plate or a 24-well culture plate) or other culture containers that are larger in size and capable of being placed into the sub-culture region 113-1 may be placed in a culture region 113 that is not arranged with the dividing member 117 or the sub-culture region 113-1 that has a relatively large size. As another example, a slide or other culture containers with a relatively small size that are capable of being placed into the sub-culture region 113-1 may be placed into the sub-culture region 113-1 with a relatively small space.

In some embodiments, the dividing member 117 may include a dividing portion 1171 and two installation portions 1172 connected with two ends of the dividing portion 1171, each of the two installation portions 1172 may include a connecting plate 1172-1 and a joint plate 1172-2, the connecting plate 1172-1 may be disposed between the dividing portion 1171 and the joint plate 1172-2, and the joint plates 1172-2 of the two installation portions 1172 may be both lapped on the heat-transfer frame 115. It should be noted that when the heat-transfer frame 115 and the installation bracket 132 are in an integrated structure, the installation of the dividing member 117 may also be regarded as the joint plate 1172-2 of one installation portion 1172 lapping over the heat-transfer frame 115 and the joint plate 1172-2 of the other installation portion 1172 lapping over the installation bracket 132.

Top surfaces of the heat-transfer frames 115 on both sides of the culture region 113 may be arranged with one or more joint slots 115-1 corresponding to one or more joint slots 115-1, respectively, the joint slots 115-1 may match the joint plates 1172-2 of the installation portion 1172, the joint plates 1172-2 of the installation portion 1172 may lap inside the joint slots 115-1, and the two oppositely disposed joint slots 115-1 on both sides of the culture region 113 may correspond to a dividing member 117. The joint slot 115-1 may play a limiting role for the dividing member 117, preventing the movement of the dividing member 117 and ensuring the stability of the sub-culture region 113-1. Moreover, a snap-fit connection of the joint slot 115-1 to the joint plate 1172-2 also makes it easy to disassemble the dividing member 117 and reduce the difficulty of adjusting the sub-culture region 113-1. In some embodiments, the two joint slots 115-1 located on the both sides of the culture region 113 are in a group, and a count of groups of joint slots 115-1 is greater than or equal to a count of the dividing members 117. In some embodiments, an extension direction of the dividing member 117 may be parallel to an edge of the culture region 113 (e.g., the X-direction shown in FIG. 4, wherein the X-direction may be a direction of the width of the heat-transfer frame 115), and the extension direction of the dividing member 117 may also be at an angle to the edge of the culture region 113. In some embodiments, a shape of the dividing member 117 may be rectilinear, or folded or other shapes, depending on the shape of the sub-culture region 113-1.

In some embodiments, two ends of the dividing member 117 may also be directly snapped between opposite sides of the heat-transfer frame 115. In some embodiments, one or more of the plurality of dividing members 117 may also be integral with the heat-transfer frame 115.

In some embodiments, the plurality of accommodation holes 134 may be disposed at intervals along a first direction (e.g., the Y-direction in FIG. 4, wherein the Y-direction may be a length direction of the heat-transfer frame 115), and the plurality of dividing members 117 may be correspondingly arranged at intervals along the first direction (e.g., the Y-direction in FIG. 4), thus the obtained sub-culture regions 113-1 may also be arranged at intervals along the first direction (e.g., the Y-direction in FIG. 4), so that the power assembly 120 installed in the accommodation hole 134 may correspond to the sub-culture regions 113-1, and the power assembly 120 may pump the culture fluid for the culture containers in the corresponding sub-culture regions 113-1, which can achieve individual perfusion culture for each sub-culture region 113-1.

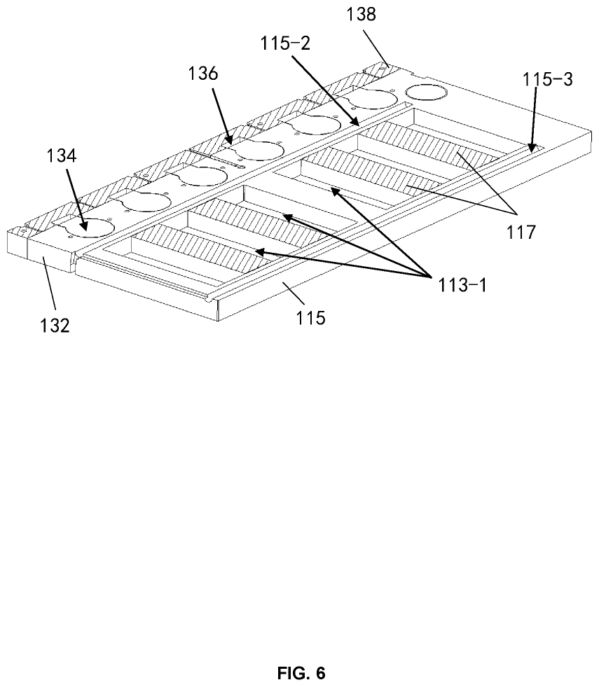

FIG. 6 is a schematic diagram illustrating an exemplary internal structure of a heat-transfer frame and a heating assembly according to some embodiments of the present disclosure. As shown in FIG. 4 and FIG. 6, in some embodiments, the heat-transfer frame 115 may be arranged with a first slot body 115-2 on a side of the heat-transfer frame 115 adjacent to the installation bracket 132, and the heat-transfer frame 115 may be arranged with a second slot body 115-3 on a side of the heat-transfer frame 115 away from the installation bracket 132. The heating element 116 may include a first heating element 116-1 and a second heating element 116-2. An extension direction of the first slot body 115-2 and an extension direction of the second slot body 115-3 may be the same as a direction (e.g., the Y-direction in FIG. 4) in which the plurality of the sub-culture regions 113-1 are arranged at intervals (i.e., the first direction), and the first heating element 116-1 may be detachably provided in the first slot body 115-2 and the second heating element 116-2 may be detachably provided in the second slot body 115-3. The first slot body 115-2 and the second slot body 115-3 may be provided in a first direction so that an arrangement direction of the first heating element 116-1 and the second heating element 116-2 is the same as an arrangement direction of the plurality of power assemblies 120 and the plurality of sub-culture regions 113-1, thereby enabling all of the plurality of sub-culture regions 113-1 to be uniformly heated. The detachable setting of the first heating element 116-1 and the second heating element 116-2 also facilitates an operator to replace the heating element 116 with a different power according to the actual situation, and provides greater flexibility in use.

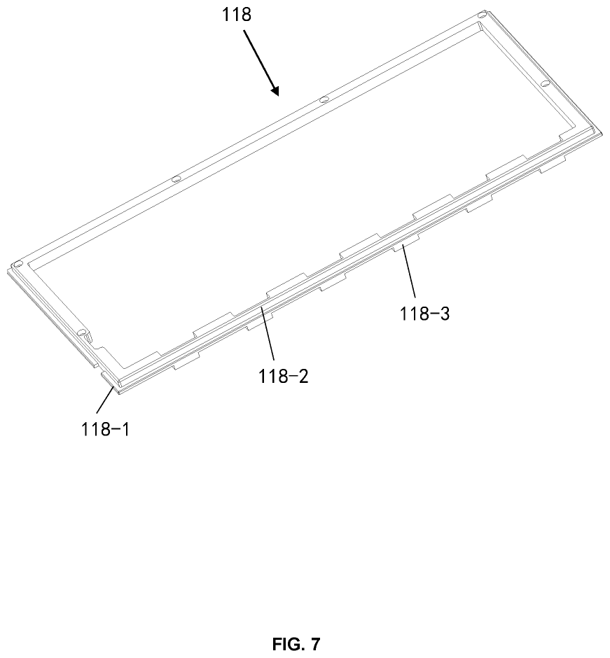

In some embodiments, the first slot body 115-2 and the second slot body 115-3 may be slots arranged within the heat-transfer frame 115 as shown in FIG. 7. The first heating element 116-1 may be inserted in the first slot body 115-2 and the second heating element 116-2 may be inserted in the second slot body 115-3. In some embodiments, the first slot body 115-2 and the second slot body 115-3 may also be slots arranged on a side of the heat-transfer frame 115, wherein the first heating element 116-1 may be disposed into and in a snap-fit connection with the first slot body 115-2, and the second heating element 116-2 may be disposed into and in a snap-fit connection with the second slot body 115-3.

Since the first heating element 116-1 is relatively closer to the installation bracket 132 relative to the second heating element 116-2, and the installation bracket 132 may absorb the heat generated by the operation of the drive portion 122 of the power assembly 120, in order to avoid an uneven temperature distribution within the culture region 113, in some embodiments, the temperature of the first heating element 116-1 may be lower than the temperature of the second heating element 116-2. Setting the temperature of the first heating element 116-1 to be lower than the temperature of the second heating element 116-2 allows the temperatures of the two sides of the culture region 113 to be closed to each other, which results in a more uniform temperature distribution within the culture region 113.

In some embodiments, by controlling the temperature of the first heating element 116-1 and the temperature of the second heating element 116-2, a sum of a heat efficiency of the first heating element 116-1 and a heat efficiency of the drive portion 122 can be equal to a heat efficiency of the second heating element 116-2, so that the temperature of the size of the culture region 113 closed to the installation bracket 132 is close to the temperature of the side of the culture region 113 away from the installation bracket 132.

In some embodiments, one end of the first heating element 116-1 and one end of the second heating element 116-2 may be connected with each other, allowing for easy disassembly and providing the power to both the first heating element 116-1 and the second heating element 116-2 for heat generation by the same energy source. In some embodiments, the first heating element 116-1 and the second heating element 116-2 may also be supplied by two different energy sources for heat generation.

FIG. 7 is a schematic diagram illustrating an exemplary structure of a cushion layer according to some embodiments of the present disclosure. As shown in FIG. 1, FIG. 2, and FIG. 7, the incubator 110 may further include an annular cushion layer 118 and an annular heat preservation cover 119, a bottom end of the annular heat preservation cover 119 may be connected with a top wall of the box body 112 via the annular cushion layer 118, and the annular cushion layer 118 may be arranged along a top end of a side wall of the box body 112. A shape of the annular cushion layer 118 may match a shape of the sidewalls of the box body 112. By placing the annular cushion layer 118 between the annular insulating cover 119 and the side wall of the box 112, the airtightness of the perfusion culture device 100 can be enhanced, resulting in a more stable temperature within the perfusion culture device 100.

In some embodiments, a material for preparing the annular heat preservation cover 119 may include an insulating material (e.g., plastic, foam board, rubber-plastic material, ceramic fibre blanket, aluminosilicate mat, silicon carbide fibre, aerogel mat, foamed cement, etc.) to enhance the heat preservation properties of the annular heat preservation cover 119.

In some embodiments, the annular heat preservation cover 119 may be hollow to increase an internal space of the incubator 110 and facilitate the installation of other components (e.g., the power assembly 120, etc.).

In some embodiments, the annular cushion layer 118 may include an annular bottom 118-1 and an annular retaining portion 118-2 arranged on the annular bottom 118-1, a size of an outer ring of the annular retaining portion 118-2 may be slightly smaller than a size of an outer ring of the annular bottom 118-1, a bottom end of the annular heat preservation cover 119 may be arranged on the annular bottom 118-1, and the annular heat preservation cover 119 may be sleeved outside of the annular retaining portion 118-2, so that the annular heat preservation cover 119 may be stably placed on the annular cushion layer 118, and the annular retaining portion 118-2 may play a limiting role for the annular heat preservation cover 119, avoiding the sliding of the annular heat preservation cover 119, and enhancing the heat preservation effect of the annular heat preservation cover 119.

In some embodiments, the size of the outer ring of the annular bottom 118-1 may match a size of an outer wall of the annular heat preservation cover 119, and an inner ring size of the annular bottom 118-1 may match an inner wall size of the annular heat preservation cover 119. In some embodiments, a bottom end of the annular heat preservation cover 119 may be provided with an annular slot (not shown in the figures) matching the annular retaining portion 118-2, and when the annular heat preservation cover 119 is placed on the annular cushion layer 118-1, the annular retaining portion 118-2 may snap to the annular slot.

In some embodiments, the annular cushion layer 118 may be connected with the top end of the sidewall of the box body 112 by bonding, threaded connection, or the like.

In some embodiments, a bottom of the annular cushion layer 118 may be arranged with protrusions 118-3, a shape and a count of the protrusions 118-3 may match a shape and a count of the wire slots 139, and a height of the protrusion 118-3 may be less than a depth of the wire slot 139 to allow for passage of the wiring of the power assembly 120. At the same time, the protrusions 118-3 may match the wire slots 139, which are also capable of limiting the wiring.

As shown in FIG. 1, FIG. 2, in some embodiments, the box body 112 may include a first heating plate 141 arranged on a top wall of the box body 112 and/or a second heating plate 142 arranged on a bottom wall of the box body 112, and the first heating plate 141 and the second heating plate 142 can improve the heating efficiency of the culture region 113, while making the temperature distribution within the culture region 113 more even.

In some embodiments, the first heating plate 141 may serve as a top wall of the box body 112, the second heating plate 142 may serve as a bottom wall of the box body 112, and a sidewall of the box body 112 may include the annular heat preservation cover 119, the annular cushion layer 118, and the heating assembly 114 connected in sequence from top to bottom. In some embodiments, when the bottom wall of the box body 112 is provided with the second heating plate 142, the heat-transfer frame 115 may not be provided with the heating element 116.

In some embodiments, the first heating plate 141 and/or the second heating plate 142 may include but are not limited to, heated glass, a plate provided with a resistive heating mechanism, a plate provided with an inductive heating mechanism, and a plate provided with an infrared heating mechanism.

In some embodiments, the first heating plate 141 and the second heating plate 142 may be visible to facilitate observation of conditions within the perfusion culture device 100. For example, the first heating plate 141 and the second heating plate 142 may be transparent. As another example, the first heating plate 141 and the second heating plate 142 may be provided with an opening for observation. In some embodiments, when the first heating plate 141 and the second heating plate 142 are transparent, the materials used to prepare the first heating plate 141 and the second heating plate 142 may include but are not limited to a transparent glass, a transparent polymer compound, or other arbitrary transparent materials. For example, the first heating plate 141 and the second heating plate 142 may be a conductive glass (e.g., Indium-Tin Oxide (ITO) conductive glass, Fluorine Doped Tin Oxid (FTO) conductive glass, etc.). When a microscope is required for observation within the perfusion culture device 100, objective lens of the microscope may be arranged below the second heating plate 142, and the light source may be arranged above the first heating plate 141. The light may pass through the first heating plate 141, the culture vessel within the culture region 113, and the second heating plate 142 in turn, and then enter the objective lens of the microscope, which can enable the operator to observe the cell culture within the perfusion culture device 100 through the eyepiece of the microscope.

In some embodiments, when there is a need to observe the inside of the perfusion culture device 100 but observation requirements are not high and the observation effect can be achieved by visual observation, the first heating plate 141 located on the top wall of the box body 112 may be made of a transparent material, and the second heating plate 142 located on the bottom wall of the box body 112 may be made of an opaque material to enable an operator to visually observe the cell culture situation inside the perfusion culture device 100 from the top of the box body 112.

In some embodiments, the first heating plate 141 and the second heating plate 142 may be made of an opaque material when there is no need for observation of the conditions within the perfusion culture device 100.

In some embodiments, the perfusion culture device 100 may further include a reservoir mechanism and a culture mechanism (not shown in the figures), the reservoir mechanism may be configured for storing the culture fluid, and the culture mechanism may be configured for carrying out the biological culture. A delivery line may be connected between the reservoir mechanism and the culture mechanism, and a portion of the delivery line may be connected with the working head 124 to allow the power assembly 120 to provide power for the delivery of the culture fluid in the delivery line.

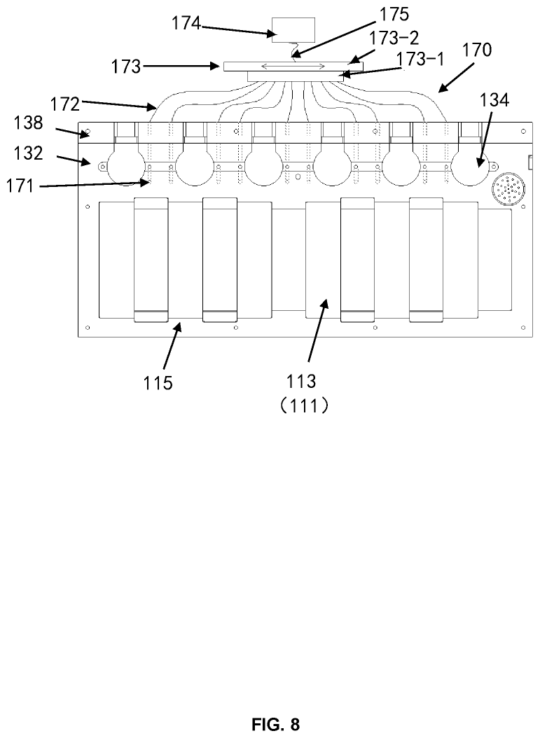

FIG. 8 is a schematic diagram illustrating an exemplary structure of a ventilation mechanism according to some embodiments of the present disclosure. Referring to FIG. 8, in some embodiments, the heat dissipation assembly 100 may further include a ventilation mechanism 170, and the ventilation mechanism may communicate with the inner cavity 111. The ventilation assembly 170 may be configured to exchange the gas with the inner cavity 111 to perform heat dissipation on the plurality of power assemblies 120.

In some embodiments, the ventilation mechanism 170 may include a plurality of ventilation holes 171, a plurality of ventilation pipes 172, one or more valves 173, and a gas pump 174. Each of the plurality of ventilation holes 171 may communicate with a corresponding ventilation pipe 172, and each of the plurality of ventilation holes 171 may form, through the corresponding ventilation pipe, a pathway with the gas pump. The one or more valves 173 may be configured to control a connection or a disconnection of the pathway. In some embodiments, each of the plurality of power assemblies 120 may be adjacent to at least one of the plurality of ventilation holes 171, and the at least one of the plurality of ventilation holes 171 may be configured to exchange gas with the inner cavity 111 for performing heat dissipation on the corresponding power assembly 120.

In some embodiments, to reduce costs, a count of the one or more valves 173 may be one, and a single valve 173 may be configured to control the connection or disconnection of all pathways. In some embodiments, in order to enhance flexibility of use, the one or more valves 173 may include a plurality of valves 173, and each of the plurality of valves valve 173 may be configured to control a connection or a disconnection of at least one pathway.

In some embodiments, the one or more valves 173 may include a switching valve. One end of each of the plurality of ventilation pipes 172 may be connected with a corresponding ventilation hole 171, and the other end of each of the plurality of ventilation pipes 172 may be connected with the switching valve. The switching valve may be connected with the gas pump 174 through a connecting tube 175. The switching valve may be configured to control a connection or a disconnection between the connecting tube 175 and the plurality of ventilation pipes 172. Through the configuration of the transfer valve, it is possible to change the ventilation pipe 172 connected to the connecting tube 175 with only one valve 173, so as to dissipate heat from the power assembly 120 near the corresponding ventilation hole 171. In the case of local overheating of a power component 120, heat dissipation can be carried out for the power component 120.

In some embodiments, the switching valve may include a guide rail 173-1 and a slider 173-2 slidably connected with the guide rail 173-1. A plurality of first connecting holes (not shown in the figure) may be arranged side by side along a sliding direction of the slider 173-2 on the guide rail 173-1. The other end (e.g., an end not connected to the ventilation hole 171) of each of the plurality of ventilation pipes 172 may be connected with a corresponding first connecting hole of the plurality of first connecting holes. The slider 173-2 may be arranged with a second connecting hole (not shown in the figure), and the second connecting hole may be connected with the connecting tube 175. In some embodiments, the switching valve may be configured to control a sliding position of the slider 173-2 to change the ventilation pipe 172 connected with the connecting tube 175 or disconnect the connection between the connecting tube 175 and the ventilation pipe 172. By controlling the sliding of the slider 173-2 relative to the guide rail 173-1, the second connecting hole may dock to a first connecting hole to connect with the first connecting hole, or dislocate with a first connecting hole to disconnect with the first connecting hole, thereby connecting or disconnecting the connecting tube 175 with different ventilation pipes 172.

In some embodiments, the switching valve may include other structures capable of changing the ventilation pipe 172 connected with the connecting tube 175 or disconnecting the connection between the connecting tube 175 and the ventilation pipe 172. For example, the switching valve may include a fixed round plate and a rotating round plate coaxially arranged with the fixed round plate. The rotating round plate may be arranged with a plurality of first connecting holes distributed in a circular pattern around a central axis of the rotating disc. Each of the plurality of first connecting holes may be connected with a corresponding ventilation pipe 172. The fixed round plate may be arranged with a second connecting hole connected with the connecting tube 175, and a distance between the second connecting hole and the central axis of the fixed round plate may be equal to a distance between any one of the plurality of first connecting holes and the central axis of the rotating round plate. By controlling a rotation of the rotating round plate relative to the fixed round plate, the second connecting hole may dock to a first connecting hole to connect with the first connecting hole, or dislocate with a first connecting hole to disconnect with the first connecting hole. As another example, the switching valve may include a gas diverter valve, and a specific channel configuration of the gas diverter valve may be designed according to a practical requirement.

In some embodiments, all of the plurality of ventilation holes 171 may be arranged on the installation bracket 132, and each of the one or more accommodation holes 134 may be positioned adjacent to at least one of the plurality of ventilation holes 171, such that the ventilation mechanism 170, when exchanging gas within the inner cavity 111, may carry away heat generated during operation of the drive portion 122 located in the accommodation hole 134. In other words, the ventilation assembly 170 may function as an air-cooling heat dissipation device, which achieves a purpose of dissipating heat from the plurality of power assemblies 120 and avoids excessive heat accumulation in the plurality of power assemblies 120 and local overheating.

In some embodiments, one end of each of the plurality of ventilation holes 171 may be arranged towards the inner cavity 111, and the other end of the plurality of ventilation holes 171 be arranged towards an outer side of the perfusion culture device 100. This configuration allows the plurality of ventilation holes 171 to be connected with the plurality of ventilation pipes 172, facilitating exchange between a gas in the inner cavity 111 and a gas outside the inner cavity 111.

In some embodiments, the plurality of ventilation holes 171 may be arranged in an L shape. One end of each of the plurality of ventilation holes 171 may match an orientation of the accommodation hole 134, and the other end of each of the plurality of ventilation holes 171 may penetrate the fixed member 138 and be positioned on a side of the box body 112. This arrangement ensures that an inflow of a gas at the plurality of ventilation holes 171 may not directly face the culture region 113, thereby minimizing an impact on a culture environment of the culture region 113.

In some embodiments, the perfusion culture device 100 may further include one or more temperature sensors (not shown in the figure) and a controller. The one or more temperature sensors may include a plurality of temperature sensors, and the plurality of temperature sensors may be arranged inside the inner cavity 111 to measure a distribution of temperatures inside the inner cavity 111. The controller may be connected with the plurality of temperature sensors and the ventilation mechanism 170 respectively through a signal connection and may be configured to control the ventilation mechanism 170 to exchange the gas with the inner cavity 111 to reduce a temperature of the inner cavity in response to determining that a temperature detected by the one or more temperature sensors exceeds a preset threshold. In some embodiments, the controller may be configured to activate, in response to determining that a local temperature detected by a temperature sensor exceeds the preset threshold, the ventilation mechanism 170, which is connected with a hose corresponding to the ventilation hole nearest to the temperature sensor, to draw out hot gas or blow in cold gas, which helps in cooling an environment and the power assembly 120 near the temperature sensor, and maintains a stability of an environment temperature for culture in the culture region 113. In some embodiments, the preset threshold may be set based on empirical knowledge or set manually. For example, the preset threshold may be the same as a heating temperature range of the heating assembly 114, such as 33° C. to 41° C. or 54° C. to 66° C., providing a temperature-stable culture environment for the perfusion culture.

In some embodiments, a ventilation hole 171 nearest to a temperature sensor, may be determined through a preset reference table. Merely by way of example, the preset reference table may include the ventilation hole 171 nearest to a specific temperature sensor. For example, the ventilation hole 171 nearest to a first temperature sensor may be a first ventilation hole.

In some embodiments, a ventilation hole 171 nearest to a temperature sensor may be determined according to a preset algorithm in a design or in an installation drawing. Merely by way of example, in the design or the installation drawing, coordinates of the temperature sensor and coordinates of two or more ventilation holes 171 adjacent to the temperature sensor may be identified. By determining a linear distance between each of the two or more ventilation holes 171 and the temperature sensor, a ventilation hole with a minimal linear distance may be designated as the ventilation hole 171 nearest to the temperature sensor. The coordinates of the temperature sensor may be coordinates of a centroid of the temperature sensor in the design or the installation drawing, and the coordinates of the ventilation hole 171 may be coordinates of the centroid of the ventilation hole 171 in the design or the installation drawing.

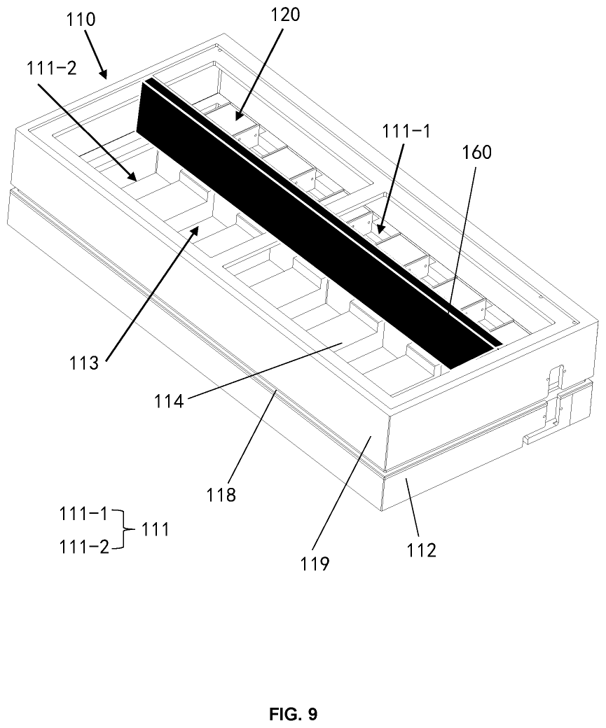

FIG. 9 is a schematic diagram illustrating another exemplary structure of a perfusion culture device according to some embodiments of the present disclosure. As shown in FIG. 9, in some embodiments, the perfusion culture device 100 may further include an separation member 160. The separation member 160 may divide the inner cavity 111 into a first chamber 111-1 and a second chamber 111-2. The power assembly 120 may be arranged in the first chamber 111-1, and a culture region (e.g., a culture region 113) of the inner cavity 111 may be arranged in the second chamber 111-2. By separating the first chamber 111-1 and the second chamber 111-2 through the separation member 160, an accumulation of the heat generated during an operation of the plurality of power assemblies 120 can be further reduced, which helps mitigate a local overheating impact on a temperature of the culture region, thereby enhancing temperature stability of the culture region and improving culture success rate. It should be noted that, in some embodiments, the first chamber 111-1 may also be considered as the inner cavity, in which the second chamber 111-2 may be regarded as an external environment of the inner cavity. Accordingly, at least a portion (e.g., the drive portions 122) of the power assembly 120 may be located outside the inner cavity.

In some embodiments, for improving the temperature stability of the culture region to improve the culture success rate, the separation member 160 may include a thermal insulating member.

In some embodiments, since the power assembly 120 primarily generates heat by the drive portion 122 during operation, to avoid the impact of excessive heat accumulation from the drive portion 122 on the temperature of the culture region, the thermal insulating member may also be arranged between the drive portion 122 and the working head 124 of the power assembly 120. The thermal insulating member may divide the inner cavity 111 into a first chamber 111-1 and a second chamber 111-2, the drive portion 122 may be arranged in the first chamber 111-1, and the working head 124 and the culture region (e.g., culture region 113) may be arranged in the second chamber 111-2. In this case, a corresponding through-hole may be provided in the separation member 160 to allow the drive portion 122 and the working head 124 to be connected via the through-holes in a transmission way.

In some embodiments, a material of the thermal insulating member may include a thermal insulating material (e.g., plastic, foam board, rubber and plastic materials, ceramic fiber blanket, aluminum silicate blanket, silicon carbide fiber, aerogel blanket, foamed cement, etc.) to enhance the thermal insulating performance of the thermal insulating member.

In some embodiments, the separation member 160 may be detachably connected with the box body 112, facilitating the removal and replacement of the separation member 160 for maintenance and reducing installation difficulty. For example, an inner side of the box body 112 may arranged with a chute, and the separation member 160 may be inserted or removed from the chute to achieve the installation and removal of the separation member 160.

In some embodiments, the separation member 160 may also be arranged with a through-hole or a through-slot (not shown in the figure) matching a delivery line. The first chamber 111-1 may be communicated with the second chamber 111-2 through the through-hole or the through-slot to provide a passage for the delivery line, allowing the delivery line driven by the plurality of power assemblies 120 to deliver a culture liquid to the culture region. In some embodiments, to facilitate the installation and removal of the separation member 160 without affecting the delivery line, a side of the separation member 160 in contact with the box body 112 (e.g., a bottom side of the separation member 160) may be provided with the through-slot.

In some embodiments, the perfusion culture device 100 may further include a gas input mechanism. The gas input mechanism may be configured to introduce a preset gas (e.g., oxygen, carbon dioxide, etc.) into the inner cavity 111, providing a suitable gas concentration environment for the culture region 113. In some embodiments, the gas input mechanism may include a gas storage tank (not shown in the figure) and a power pump, and the power pump may be connected with the gas storage tank through a pipeline. The power pump may transport the preset gas from the gas storage tank to the inner cavity 111 through the plurality of pipes.

In some embodiments, the perfusion culture device 100 may further include a gas concentration detection mechanism (not shown in the figure), which may be configured to sense a concentration of the preset gas in the inner cavity 111, thereby monitoring the gas concentration environment in the inner cavity 111 in real-time. In some embodiments, the gas input mechanism and the gas concentration detection mechanism may be connected with the controller. The controller may be configured to control whether the gas input mechanism introduces the preset gas into the inner cavity 111 based on the concentration of the preset gas sensed by the gas concentration detection mechanism. Merely for example, when the preset gas is carbon dioxide, the controller may firstly control the gas input mechanism to introduce carbon dioxide into the inner cavity 111. Simultaneously, the gas concentration detection mechanism may continuously monitor the carbon dioxide concentration in the inner cavity 111. When a detected carbon dioxide concentration reaches or exceeds a preset level (e.g., 4.95%), the controller may instruct the gas input mechanism to stop introducing carbon dioxide. As carbon dioxide may be consumed during cell perfusion culture, the carbon dioxide concentration in the inner cavity 111 may gradually decrease. When the gas concentration detection mechanism detects that the carbon dioxide concentration in the inner cavity 111 is below the preset level (e.g., 4.95%), the controller may again instruct the gas input mechanism to introduce carbon dioxide.

In some embodiments, the gas concentration detection mechanism may include an exhaust port, a suction pump (e.g., a diaphragm pump), an exhaust pipe, and a concentration detector. The exhaust port may be arranged on the box body 112, and the suction pump may be arranged at the exhaust port. One end of the exhaust pipe may be connected with the exhaust port, and the other end of the exhaust pipe may be connected with the concentration detector. In some embodiments, when the gas input mechanism includes two or more power pumps, some of the two or more power pumps may be connected with the gas storage tank to transport the preset gas into the inner cavity 111. The remaining of the two or more power pumps may be directly connected with an external atmosphere, serving as exhaust pump(s) to expel the gas from the inner cavity 111. In this case, corresponding pipe(s) may act as exhaust pipe(s), and the concentration detector may be disposed at a corresponding power pump.

The beneficial effects in the embodiments of the present discourse may include but are not limited to the followings. (1) By setting the power assembly inside the perfusion culture device, the power assembly is installed on the heat dissipation assembly, so that the heat from the power assembly can be rapidly transferred, thereby preventing local overheating in the culture region from affecting the cell culture. (2) By setting the installation bracket on one side of the heat-transfer frame, the heat generated by the drive portion of the power assembly is absorbed by the installation bracket and then evenly dispersed into the culture region through the heat-transfer frame, so that the heat is recovered and used to maintain the temperature of the culture region, thereby saving energy. (3) By providing a dividing member made of a heat-transfer material in the culture region, the culture region is divided into a plurality of sub-culture regions, which not only improves the scale of culture of organisms, but also maintains the temperature of the sub-culture regions uniformly and stably. (4) By arranging a heating assembly around on the upper side of the bottom wall of the box body, the temperature in the culture region is easier to control. (5) By providing heating elements inside the heat-transfer frame on one side of the power assembly and on the opposite side away from the power assembly, the heat transferred through the dividing member is more uniform, which in turn makes the temperature in the culture region more uniform. It should be noted that different embodiments may produce different beneficial effects, and in different embodiments, the beneficial effects that may be produced may be any one or a combination of any one or more of the above, or any other beneficial effect that may be obtained.

The basic concepts have been described above, obviously, to those skilled in the art, the above detailed disclosure is intended as an example only and does not constitute a limitation of the present disclosure. Although there is no clear explanation here, those skilled in the art may make various modifications, improvements, and modifications of present disclosure. This type of modification, improvement, and corrections are recommended in present disclosure, so the modification, improvement, and the amendment remain in the spirit and scope of the exemplary embodiment of the present disclosure.

At the same time, present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of present disclosure are not necessarily all referring to the same embodiment. Further, certain features, structures, or features of one or more embodiments of the present disclosure may be combined.

Moreover, unless the claims are clearly stated, the sequence of the present disclosure, the use of the digital letters, or the use of other names is not configured to define the order of the present disclosure processes and methods. Although some examples of the disclosure currently considered useful in the present disclosure are discussed in the above disclosure, it should be understood that the details will only be described, and the appended claims are not limited to the disclosure embodiments. The requirements are designed to cover all modifications and equivalents combined with the substance and range of the present disclosure. For example, although the implementation of various components described above may be embodied in a hardware device, it may also be implemented as a software only solution, e.g., an installation on an existing server or mobile device.

Similarly, it should be noted that in order to simplify the expression disclosed in the present disclosure and help the understanding of one or more embodiments, in the previous description of the embodiments of the present disclosure, a variety of features are sometimes combined into one embodiment, drawings or description thereof. However, this disclosure method does not mean that the characteristics required by the object of the present disclosure are more than the characteristics mentioned in the claims. Rather, claimed subject matter may lie in less than all features of a single foregoing disclosed embodiment.

In some embodiments, numbers expressing quantities of ingredients, properties, and so forth, configured to describe and claim certain embodiments of the application are to be understood as being modified in some instances by the term “about,” “approximate,” or “substantially”. Unless otherwise stated, “approximately”, “approximately” or “substantially” indicates that the number is allowed to vary by ±20%. Accordingly, in some embodiments, the numerical parameters used in the specification and claims are approximate values, and the approximate values may be changed according to characteristics required by individual embodiments. In some embodiments, the numerical parameters should be construed in light of the number of reported significant digits and by applying ordinary rounding techniques. Although the numerical domains and parameters used in the present disclosure are configured to confirm its range breadth, in the specific embodiment, the settings of such values are as accurately as possible within the feasible range.

For each patent, patent application, patent application publication and other materials referenced by the present disclosure, such as articles, books, instructions, publications, documentation, etc., hereby incorporated herein by reference. Except for the application history documents that are inconsistent with or conflict with the contents of the present disclosure, and the documents that limit the widest range of claims in the present disclosure (currently or later attached to the present disclosure). It should be noted that if a description, definition, and/or terms in the subsequent material of the present disclosure are inconsistent or conflicted with the content described in the present disclosure, the use of description, definition, and/or terms in this manual shall prevail.

Finally, it should be understood that the embodiments described herein are only configured to illustrate the principles of the embodiments of the present disclosure. Other deformations may also belong to the scope of the present disclosure. Thus, as an example, not limited, the alternative configuration of the present disclosure embodiment may be consistent with the teachings of the present disclosure. Accordingly, the embodiments of the present disclosure are not limited to the embodiments of the present disclosure clearly described and described.

What is claimed is:

1. A perfusion culture device, comprising an incubator, a heat dissipation assembly, and a plurality of power assemblies; wherein the incubator includes a box body and an inner cavity surrounded by the box body, the plurality of power assemblies are disposed in the inner cavity, the heat dissipation assembly is fixed on the box body, and the plurality of power assemblies are arranged on the heat dissipation assembly.

2. The perfusion culture device of claim 1, wherein the plurality of power assemblies include a drive portion and a working head, the heat dissipation assembly includes an installation bracket and one or more accommodation holes arranged on the installation bracket, the working head is arranged on the installation bracket, the drive portion is arranged in the accommodation holes, and at least a portion of inner wall of the accommodation holes is adhered to the drive portion.

3. The perfusion culture device of claim 2, wherein the heat dissipation assembly further includes one or more fixing members, the one or more fixing members are connected with the installation bracket, each of the one or more fixing members is provided with a wire slot that communicates with the one or more accommodation holes.

4. The perfusion culture device of claim 2, wherein the box body further includes a heating assembly, the heating assembly includes a heat-transfer frame and a heating element, the heating element is arranged on the heat-transfer frame, the installation bracket is arranged outside the heat-transfer frame, the installation bracket is arranged adjacent to the heat-transfer frame, and the heat-transfer frame is connected with a bottom wall of the box body to form a culture region.

5. The perfusion culture device of claim 4, wherein the installation bracket is connected with the bottom wall of the box body; the installation bracket and the heat-transfer frame form a side wall of the box body; and the installation bracket and the heat-transfer frame are in an integrated structure.

6. The perfusion culture device of claim 4, wherein the heating assembly further includes one or more dividing members, the one or more dividing members are connected with the heat-transfer frame, and the one or more dividing members are arranged within the culture region to divide the culture region into a plurality of sub-culture regions.

7. The perfusion culture device of claim 6, wherein the one or more dividing members include a dividing portion and two installation portions respectively connected with two ends of the dividing portion, each of the installation portions includes a connecting plate and a joint plate, the connecting plate is disposed between the dividing portion and the joint plate, and the joint plates of the two installation portions are lapped onto the heat-transfer frame.

8. The perfusion culture device of claim 6, wherein
a plurality of the accommodation holes are arranged at intervals along a first direction, and the plurality of sub-culture regions are arranged at intervals along the first direction;
a first slot body is provided on a side of the heat-transfer frame closed to the installation bracket, and a second slot body is provided on a side of the heat-transfer frame away from the installation bracket;
an extension direction of the first slot body and the second slot body are the same as the first direction; and
a first heating element is detachably provided in the first slot body, and a second heating element is detachably provided in the second slot body.

9. The perfusion culture device of claim 5, wherein
the incubator includes an annular cushion layer and an annular heat preservation cover, a top end of the annular heat preservation cover is connected with a top wall of the box body, and the annular cushion layer is arranged along a top end of a side wall of the box body;
the annular cushion layer includes an annular bottom and an annular retaining portion disposed on the annular bottom, a size of an outer ring of the annular retaining portion is smaller than a size of an outer ring of the annular bottom, a bottom end of the annular heat preservation cover is arranged on the annular bottom, and the annular heat preservation cover is sleeved outside the annular retaining portion.

10. The perfusion culture device of claim 1, wherein the box body includes a first visible heating plate provided on a top wall of the box body and/or a second visible heating plate provided on a bottom wall of the box body.

11. The perfusion culture device of claim 1, wherein the heat dissipation assembly further includes a ventilation mechanism, the ventilation mechanism communicates with the inner cavity, and the ventilation mechanism is configured to exchange gas with the inner cavity for performing heat dissipation on the plurality of power assemblies.

12. The perfusion culture device of claim 1, wherein
the ventilation mechanism includes a plurality of ventilation holes, a plurality of ventilation pipes, one or more valves, and a gas pump, each of the plurality of ventilation holes communicates with a corresponding ventilation pipe of the plurality of ventilation pipes, each of the plurality of ventilation holes forms, through the corresponding ventilation pipe, a pathway with the gas pump, and the one or more valves are configured to control a connection or a disconnection of the pathway; and
each of the plurality of power assemblies is adjacent to at least one of the plurality of ventilation holes, and the at least one of the plurality of ventilation holes is configured to exchange gas with the inner cavity for performing heat dissipation on the corresponding power assembly.

13. The perfusion culture device of claim 12, wherein the heat dissipation assembly includes an installation bracket and one or more accommodation holes arranged on the installation bracket, and the plurality of power assemblies are installed in the one or more accommodation holes;
the plurality of the ventilation holes are arranged on the installation bracket, each of the one or more accommodation holes is adjacent to at least one of the plurality of ventilation holes, one end of each of the plurality of ventilation holes is arranged towards the inner cavity, and the other end of each of the plurality of ventilation holes is arranged towards an outer side of the perfusion culture device.

14. The perfusion culture device of claim 12, wherein the one or more valves include a switching valve, one end of each of the plurality of ventilation pipes is connected with a corresponding ventilation hole of the plurality of ventilation holes, the other end of each of the plurality of ventilation pipes is connected with the switching valve, the switching valve is connected with the gas pump through a connecting tube, and the switching valve is configured to control a connection or a disconnection between the connecting tube and the plurality of ventilation pipes.

15. The perfusion culture device of claim 14, wherein the switching valve includes a guide rail and a slider slidingly connected with the guide rail, a plurality of first connecting holes are arranged side by side along a sliding direction of the slider on the guide rail, the other end of each of the plurality of ventilation pipes is connected with a corresponding first connecting hole of the plurality of first connecting holes, the slider is arranged with a second connecting hole, and the second connecting hole is connected with the connecting tube, the switching valve is configured to control a sliding position of the slider to change the ventilation pipe connected with the connecting tube or disconnect the connection between the connecting tube and the ventilation pipe.

16. The perfusion culture device of claim 11, further comprising one or more temperature sensors and a controller, wherein the one or more temperature sensors are arranged inside the inner cavity, the controller is connected with the one or more temperature sensors and the ventilation mechanism through a signal connection, and the controller is configured to control the ventilation mechanism to exchange the gas with the inner cavity to reduce a temperature of the inner cavity in response to determining that a temperature detected by the one or more temperature sensors exceeds a preset threshold.

17. The perfusion culture device of claim 1, wherein the perfusion culture device further includes a gas input mechanism configured to introduce a preset gas to the inner cavity.

18. The perfusion culture device of claim 1, wherein the perfusion culture device further includes a gas concentration detection mechanism configured to detect a gas concentration in the inner cavity.

19. The perfusion culture device of claim 1, further comprising a separation member detachably connected with the box body, wherein the separation member divides the inner cavity into a first chamber and a second chamber, the plurality of power assemblies are arranged in the first chamber, and a culture region of the inner cavity is arranged in the second chamber.

20. The perfusion culture device of claim 19, wherein the separation member include a thermal insulating member.
